(19) United States
(12) Patent Application Publication
(10) Pub. No.: US 2009/0149551 A1
(43) Pub. Date: Jun. 11, 2009

(54) OXIDIZED cis-TERPENONE AND ITS USE AS A CHEMOPROTECTIVE AND ANTI-MALARIAL AGENT

(76) Inventors: Qibing Zhou, Glen Allen, VA (US); Ghislaine Mayer, Richmond, VA (US); Lin Zhang, Richmond, VA (US); Jennifer K. Stewart, Richmond, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/196,352

(22) Filed: Aug. 22, 2008

Related U.S. Application Data
(60) Provisional application No. 61/012,080, filed on Dec. 7, 2007.
(60) Provisional application No. 61/073,121, filed on Jun. 17, 2008.

(51) Int. Cl. A61K 31/122; C07C 49/00; A61P 31/00

(52) U.S. Cl. 514/680; 568/326

(57) ABSTRACT

4b,5,6,7,8,8a-cis-hexahydro-2-hydroxy-4b,8,8a-trimethylphenanthren-9,10-dione (OHCT) is a fully oxidized compound based on the 2-hydroxy-cis-terpenone (HCT). Because OHCT is fully oxidized, it is more stable and less likely to damage cells or cell constituents such as DNA. OHCT has antimalarial activity, noncompetitive inhibitory activity against carcinogenic conversion of aflotoxin B1 (AFB1) to exo-AFB1-epoxide thus inihibiting AFB1 induced cellular toxicity, and inhibitory activity against TCDD induced cellular toxicity.

### CROSS-REFERENCE TO RELATED APPLICATIONS

This application claims priority to U.S. Provisional Patent Application Ser. No. 61/012,080, filed Dec. 7, 2007, and to U.S. Provisional Patent Application Ser. No. 61/073,121 filed Jun. 17, 2008, and the complete contents these applications are herein incorporated by reference.

This invention was made using funding from the National Science Foundation under grant MCB 0131419. The U.S. Government may have certain rights to use this invention.

### FIELD OF THE INVENTION

The invention is generally related to the synthesis of an oxidized form of cis-terpenone and its use as a chemoprotective agent against aflatoxin B1 induced cytotoxicity and its use in killing Plasmodium falciparum.

### BACKGROUND

Aflatoxin B₁ (AFB1) was first discovered in the 1960s and since then, numerous studies have consistently shown that populations with hepatitis B and C infections have a much higher risk of hepatocellular carcinoma with dietary exposure of AFB1. AFB1 is a major mycotoxin produced by the fungus A. fiavus and is commonly found as a crop contaminant. In past decades, several effective chemoprevention agents, including oltipraz and chlorophyllins, have been developed and showed promising results in clinical trials. Unfortunately, adverse effects were the major concern for oltipraz, while the low complexing constant between the dietary supplement chlorophyllin and AFB1 required high dosages of chlorophyllin. In liver, AFB1 is metabolically converted by P450s to the carcinogenic exo-AFB1-epoxide, which forms mutagenic AFB1-DNA adducts. Besides DNA modification, reactive oxygen species are generated with the metabolic activation of AFB1, causing additional hepatotoxicity.

The detailed metabolic conversion of AFB1 has been established by Guengerich and co-workers through meticulous kinetic studies (see, for example, Shimada, T., and Guengerich, F. P. (1989)—Evidence for cytochrome P-450_{NF}, the nifedipine oxidase, being the principal enzyme involved in the bioactivation of aflatoxins in human liver—Proc. Natl. Acad. Sci. U.S.A. 86, 462-465; Guengerich, F. P., and Kim, D. H. (1990)—In vitro inhibition of dihydropyridine oxidation and aflatoxin B₁ activation in human liver microsomes by naringenin and other flavonoids—Carcinogenesis, 11, 2275-2279; Raney, K. D., Shimada, T., Kim, D. H., Groopman, J. D., Harris, T. M., and Guengerich, F. P. (1992)—Oxidation of aflatoxins and sterigmatocystin by human liver microsomes: significance of aflatoxin Q₁ as a detoxication product of aflatoxin B₁—-Chem. Res. Toxicol. 5, 202-210; Ueng, Y. F., Shimada, T., Yamazaki, H., and Guengerich, F. P. (1995)—Oxidation of aflatoxin B₁ by bacterial recombinant human cytochrome P450 enzymes—Chem. Res. Toxicol. 8, 218-225; Johnson, W. W., Harris, T. M., and Guengerich, F. P. (1996)—Kinetics and mechanism of hydrolysis of aflatoxin B₁ exo-8,9-epoxide and rearrangement of the dihydrodiol—J. Am. Chem. Soc. 118, 8213-8220; Johnson W. W., and Guengerich, F. P. (1997)—Reaction of aflatoxin B₁-exo-8,9-epoxide with DNA: Kinetic analysis of covalent binding and DNA induced hydrolysis—Proc. Natl. Acad. Sci. U.S.A. 94, 6121-6125; Gengerich, F. P., Johnson, W. W., Shimada, T., Ueng, Y. F., Yamazaki, H., and Langouet, S. (1998)—Activation and detoxication of aflatoxin B₁—Mut. Res. 402, 121-128; and Guengerich, F. P. (2003)—Cytochrome P450 oxidations in the generation of reactive electrophiles: epoxidation and related reactions—Arch. Biochem. Biophys. 409, 59-71).

Initially, it was proposed that P450 1A2 was responsible for AFB1 activation at low concentrations and P450 3A4 at high concentrations. However, consistent results indicated that P450 3A4 was the dominant activation enzyme at all concentrations. Furthermore, Guengerich and coworkers revealed that the metabolism of AFB1 with P450 3A4 produces only the carcinogenic exo-AFB1-epoxide and the detoxification product aflatoxin Q₁. In contrast, P450 1A2 yields primarily aflatoxin M₁, and low amounts of aflatoxin Q₁ and equal ratios of exo- and endo-AFB1-epoxides. Recently, the relative contribution of P450s to the carcinogenic conversion of AFB1 was confirmed as 3A4>3A5>>1A2 (see, Kamdem, L. K., Meineke, I., Gödtel-Armbrust, U., Brockmoller, J., and Wojnowski, L. (2006) Dominant Contribution of P450 3A4 to the Hepatic Carcinogenic Activation of Aflatoxin B₁. Chem. Res. Toxicol. 19, 577-586).

It would be advantageous to have a better understanding of chemoprotective mechanisms of action for agents that protect against AFB1 induced cytotoxicity, and to identify agents that are stable, water soluble, non-toxic to the human or other mammalian liver, and effective against AFB1 induced cytotoxicity.

Malaria affects millions of people worldwide. The causative agent of the most severe form of malaria is the parasite Plasmodium falciparum. Malaria has been commonly treated with artemisinin and chloroquine; however, parasites that are resistant to these compounds have evolved. It would be advantageous to have new agents which are not toxic to humans or animals, but which kill all life-cycle stages of Plasmodium falciparum including the gametocyte stage that is responsible for infecting mosquitos which transmit the disease.

### SUMMARY

A compound of the structure

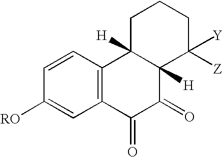

where R is a hydrogen and Y and Z are both methyls (hereinafter OHCT) has been identified and synthesized. OHCT is the fully oxidized version of the terpenone 2-hydroxy-cis-terpenone (HCT). Because OHCT is fully oxidized, it is more stable and less likely to damage cells or cell constituents such as DNA. OHCT has antimalarial activity, noncompetitive inhibitory activity against carcinogenic conversion of aflatoxin B1 (AFB1) to exo-AFB1-epoxide thus inhibiting AFB1 induced cellular toxicity, and inhibitory activity against TCDD induced cellular toxicity. OHCT can be administered to subjects in need of prophylactic prevention or treatment of malarial infections caused by Plasmodium falciparum. OHCT has been found to kill Plasmodium falciparum in any life cycle stage and to kill parasites which are resistant to two commonly used antimalarial agents. OHCT also has been demonstrated to inhibit aflatoxin B1 induced cytotoxicity by non-competitive inhibition with P450 enzymes such as 3A4, and to inhibit TCDD induced cytotoxicity by inhibition of P450 enzymes such as 1A1 and 1B1. Inhibition of P450 1B1 is an anti-carcinogennesis strategy because it activates many carcinogens including polycyclic aromatic hydrocarbons (benzo[a]pyrene), heterocyclic amines, aromatic amines, nitropolycyclic hydrocarbons and estrogens (see, Guengerich, F. P.; Chun, Y.-J.; Kim, D.; Gillam, E. M. J.; Shimada, T. (2003) Cytochrome P450 1B1: a target for inhibition in anticarcinogenesis strategies. Mutation Research, 523-524, 173-182). OHCT, or conjugates or derivatives thereof, can be administered to subjects for preventing either or both aflatoxin B1 and other carcinogens.

### DESCRIPTION OF THE DRAWINGS

FIGS. 1A-1L are graphs showing HPLC analysis of the inhibitory effects of HCT on the metabolic conversion of AFB1 with pooled human liver microsomes. Experimental conditions: (a)-(d) and (h)-(l) were carried out at 37° C. for 90 min in 100 mM phosphate buffer (pH 7.4), 1.3 mM NADP⁺, 3.3 mM MgCl₂, 3.3 mM glucose-6-phosphate, 0.3 unit/mL glucose-6-phosphate dehydrogenase, 20 mM GSH, 2 mM EDTA, 0.1 mg/mL GST, 0.4 mg/mL liver microsomes, 50 μM AFB1 and 1% DMSO; (e) same as (a) without liver microsomes at 37° C. for 90 min; (f) 40 μM exo-AFB1-epoxide in 100 mM phosphate buffer (pH 7.4) and 1% DMSO at 37° C. for 90 min; (g) 40 μM exo-AFB1-epoxide in 100 mM phosphate buffer (pH 7.4), 20 mM GSH, 2 mM EDTA, 0.1 mg/mL GST and 1% DMSO at 37° C. for 90 min.

FIG. 2 is a bar graph showing the inhibitory effects of HCT on the activity of purified P450 3A4 by Vivid® activity assay with a NADPH regenerating system. All experiments were carried out in 100 mM phosphate buffer (pH 8.0), 3.3 mM glucose-6-phosphate, 0.3 unit/mL glucose-6-phosphate dehydrogenase, 1.0 mM NADP⁺ and 2 μM substrate (DBOMF). The rates of the reactions were derived from the change of the fluorescent intensity (excitation/emission: 485/535 nm) in the initial 8 min. Each bar represents the mean and the standard deviation of triplicate experiments and the rate obtained with substrate was used as the reference in the percentage calculation.

FIG. 3a-3e are graphs of HPLC analysis showing the spontaneous oxidation of HCT under aqueous buffered conditions. Experimental Conditions: (a)-(c) were carried out in 100 mM phosphate buffer (pH 7.4) and 10% acetontrile; (d)-(e) 100 mM phosphate buffer (pH 7.4), 1.3 mM NADP⁺, 3.3 mM MgCl₂, 3.3 mM glucose-6-phosphate, 0.3 unit/mL glucose-6-phosphate dehydrogenase, 20 mM GSH, 2 mM EDTA, 0.1 mg/mL GST, 0.4 mg/mL liver microsomes, 50 μM AFB1 and 1% DMSO. The HPLC signals were observed at 280 nm.

FIGS. 4a-4d are line and bar graphs showing the effects of OHCT against AFB1 metabolism, P450 3A4 enzyme activity and AFB1 cytotoxicity. (a)-(b) HPLC analysis of the inhibitory effects on the metabolic conversion of AFB1 with pooled human liver microsomes. The experiments were carried out similarly as in FIG. 1a; (c) The inhibitory effect on the activity of purified P450 3A4 by Vivid® activity assay with a NADPH regenerating system. The experiment was carried out similarly as in FIG. 2; (d) Chemo-protection against AFB1-induced cytotoxicity. HepG2 cells were co-treated with AFB1 (2 μM) and OHCT or ketoconazole and then incubated for 72 h. Cell viability was measured with the MTT assay. The percentage of viable cells was based on cells treated with DMSO only. Each bar represents the mean ±SD of four replicates. The data are representative of three independent experiments. *P<0.01 compared to treatment with AFB1 only by one way ANOVA and Dunnett's test.

FIG. 5 is a graph showing mixed inhibition of P450 3A4 enzyme activity with OHCT by the Lineweaver-Burk plot. All experiments were carried out in 100 mM phosphate buffer (pH 8.0) with a NADPH regenerating system. The rates of the reactions were derived from the change of the fluorescent intensity (excitation/emission: 485/535 nm) in the initial 4 min. Each data point represents the mean and the standard deviation of triplicate experiments.

FIG. 6 is a schematic drawing illustrating chemoprotection of HCT against AFB-1 induced cytotoxicity and TCDD-induced P450-1 activity in liver HepG2 cells.

FIG. 7 is a schematic drawing showing carcinogenic activation of AFB1 and subsequent GSH addition and hydrolysis

FIG. 8 is a schematic drawing illustrating that OHCT is the spontaneous oxidation product of HCT.

FIG. 9 is a table showing the in vitro antimalarial activities of OHCT against chloroquine-resistant P. falciparum clone Dd2. The chloroquine resistant clone of P. falciparum, Dd2, was killed by OHCT in a concentration dependnt manner 24 hr following treatment. The early ring, trphozoite and schizont stages, which constitute the intraerthrocytic life cycle of the parasite, were equally inhibited.

FIG. 10 is a bar graph presenting the data of FIG. 9 in a graphical format.

FIG. 11 are enlarged microscopic images that show that 2 μM OHCT, but not 2 μM chloroquine, was active against the gametocyte forms of the parasite responsible for infecting mosquitoes and thus transmitting the disease.

FIG. 12 is a table demonstrating the time course of OHCT action on P. falciparum clone Dd2. Parasitemia decreased by 50% following treatment with OHCT for 8 hours.

FIG. 13 is a schematic drawing of OHCT illustrating sites for conjugation and/or modification to form OHCT conjugates or derivatives.

### DETAILED DESCRIPTION

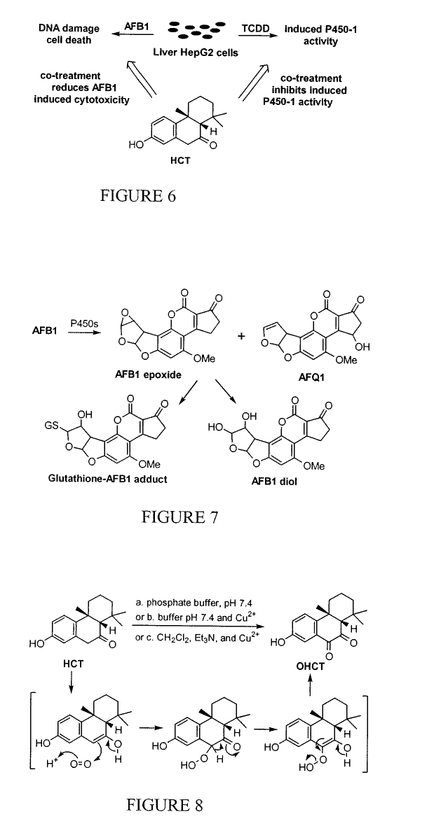

Our investigative group recently reported the protective effect of 2-hydroxy-cis-terpenone (HCT) against AFB1-induced cytotoxicity in human HepG2 liver cells (Zhou et al. Chem. Res. Toxicol. 2006, 19, 1415-1419); however, the mechanism of action was not clear. FIG. 6 illustrates chemoprotection of HCT against AFB1-induced cytotoxity and TCDD-induced P450-1 activity in liver HpG2 cells. Co-treatment of liver HepG2 cells with AFB1 and HCT resulted in reduced AFB1 induced cytotoxicity including DNA damage and cell death. Co-treatment of liver HepG2 cells with HCT and TCDD (2,3,7,8 tetrachlorodibenzo-p-dioxin) inhibited induced P450-1 activity.

HCT was developed as a stereoisomer of the possible metabolic precursor of natural terpene quinine methide analogs. In addition to the protection against AFB1, HCT also exhibits an inhibitory effect on the activity of P450 1A/1B induced by TCDD in liver cells. Therefore, we investigated the chemoprotective mechanism with liver nicrosomes and purified P450 3A4 enzyme. As will be discussed in more detail below, HCT showed effective inhibition of the metabolic conversion of AFB1 in liver microsomes at 40 μM and more importantly, the inhibition of the carcinogenic exo-AFB1-epoxide formation from AFB1. Further study indicated the direct inhibition of purified P450 3A4 enzyme activity by HCT with an IC₅₀ value of 20 μM. Under aqueous conditions, HCT was found to be slowly converted to an oxidized product, 4b,5,6,7,8,8a-cis-Hexahydro-2-hydroxy-4b,8,8a-trimethylphenanthren-9,10-dione (OHCT), which previously has not been described. A synthesis procedure for producing OHCT was developed. It was found that OHCT exhibits similar inhibitory effects on both P450 3A4 and the metabolic conversion and carcinogenic activation of AFB1 with liver microsomes as those of HCT. Enzyme mechanism studies revealed that OHCT acted as a mixed inhibitor of P450 3A4 with Kᵢ and Kᵢ′ at 17.6±5.6 and 7.6±1.5 μM, respectively. Finally, OHCT showed no cytotoxicity at 60 μM in HepG2 liver cells and effective chemoprotection at 40 and 60 μM against AFBL (2 μM) induced cytotoxicity. These results were compared to those with ketoconozole, a clinically approved drug that is used despite problems with liver toxicity. Ketoconazole alone exhibited 20% HepG2 cell mortality at 20 μM. Chemoprotection with ketoconazole against 2 μM AFB1 in HepG2 cells was observed at 10 and 20 μM, which were much higher than the 1 μM concentration used in the inhibitory assays of P450 3A4 activity and AFB1 metabolism with liver microsomes.

Furthermore, it was found that OHCT killed all stages of Plasmodium falciparum, and killed parasites that are resistant to chloroquine (a drug that is no longer used in most areas because of resistance) and to artemisinin (a drug that is used to treat malaria in many areas of the world). As an antimalarial, OHCT has the following advantages: it has a structure unlike that of known antimalarials; it has a mechanism of action unlike that of chloroquine and artemisinin, as indicated by the ability to kill parasites resistant to these drugs; it interacts with multiple targets in the human liver and lung cells; it has a structure permitting facile conjugation with other chemical entities; inhibition of human liver P450 enzyme activity, thus reducing metabolism of animalarials; and unlike chloroquine, OHCT is active against the gametocyte forms of Plasmodium responsible for infecting mosquitoes and transmitting disease.

### Experimental Procedures

All chemicals were purchased from Fisher Scientific (Pittsburgh, Pa.) or Sigma-Aldrich (Milwaukee, Wis.) and used without further purification. The IR spectrum was recorded with a Nicolet Avatar 320 FT-IR from Thermo Scientific (Waltham, Mass.). The NMR spectra were obtained with Variant NMR spectrometers (Walnut Creek, Calif.). HPLC analysis was carried out on a Jasco 2000 series station (Easton, Md.) with a Microsorb MV C18 column (250×2.1 mm, 8 μm) from Varian at a flow rate of 1 mL/min using a gradient condition (CH₃CN in 10 mM triethylammonium acetate buffer (pH 5.5): 10% for 5 min, then from 10% to 70% over 30 min, and then from 70% to 100% over 5 min). Electrospray ionization mass spectroscopy (ESI-MS) analysis was carried out with a Q-TOF2 Micromass (Manchester, UK). Human glutathione S-transferase (GSH) from placenta was obtained from Sigma-Aldrich. Pooled human liver microsomes were purchased from BD Biosciences (Lot Number: 36170, Woburn, Mass.), and the activities of several P450s were pre-determined by the supplier (1A2 at 570 and 3A4 at 5900 pmol/min per mg protein, respectively). Vivid® CYP450 3A4 Green Screen kit was obtained from Invitrogen (Carlsbad, Calif.), and the fluorescent intensity of the activity assay was recorded with a Wallac 1420 Victor² Multilabel Counter from Perkin Elmer (Waltham, Mass.). HCT was synthesized as reported previously (31) and exo-AFB1-epoxide was received as a gift.

Liver Microsomes and P450 3A4 Studies with HCT. The metabolic conversion of AFB1 was carried out in 100 mM phosphate buffer (pH 7.4), 1.3 mM NADP⁺, 3.3 mM MgCl₂, 3.3 mM glucose-6-phosphate, 0.3 unit/mL glucose-6-phosphate dehydrogenase, 20 mM reduced glutathione (GSH), 2 mM EDTA, 0.1 mg/mL GST, 0.4 mg/mL liver microsomes, 50 μM AFB1 and 1% DMSO. The final reaction volume was 100 μL. Prior to each assay, solutions of HCT in acetonitrile were added to the tubes and lyophilized to dryness by vacuum at 5 millitorr for 15 min, which afforded a concentration of 10, 20, or 40 μM upon the addition of the assay solutions. Stock solutions of ketoconazole and α-naphthoflavone were prepared in acetonitrile, and cholesterol was dissolved in ethanol. For the assay, reaction solutions were incubated at 37° C. for 90 min and then 0.5 M phosphoric acid (5 μL) was added. The resulting solutions were mixed and centrifuged at 16,000×g for 4 min, and the supernatants were collected and stored at −15° C. HPLC analysis of these samples was carried out within 5 h and no side reaction was observed within the storage time. Control experiments included AFB1 without liver microsomes, direct hydrolysis of exo-AFB1-epoxide, and trapping of exo-AFB1-epoxide with GSH and GST.

For the identification of the AFB1-diol, exo-AFB1-epoxide (40 μM) was incubated at 37° C. for 90 min in the phosphate buffer solution (100 mM, pH 7.4, 100 μL), and analyzed with both HPLC and MS analyses as described as above. The formation of GSH-AFB1 adduct was achieved in the phosphate buffer solution (100 MM, pH 7.4, 100 μL) with 20 mM GSH, 2 mM EDTA, 0.1 mg/mL GST and 40 μM exo-AFB1-epoxide. The metabolic conversion of AFB1 by liver microsomes with or without HCT using NADPH were carried out in 100 mM phosphate buffer (pH 7.4), 1.3 mM NADPH, 3.3 mM MgCl₂, 0.4 mg/mL liver microsomes, 50 μM AFB1 and 1% DMSO. The HPLC analysis was carried out similarly as described above.

The Vivid® P450 3A4 activity assay was carried out according to the manufacturer's protocol. Briefly, water (30 μL) and solutions of HCT (10 μL) were added to wells of a 96-well black plate. Master pre-mix solutions (50 μL) containing recombinant human P450 3A4 and rabbit NADPH P450 reductase were then added, and the resulting solutions were equilibrated at room temperature for 20 min. Finally, 10 μL of mixed NADP⁺ and DBOMF substrate solutions were added, and the fluorescence intensity over time was recorded with a fluorometer at the excitation and emission wavelengths of 485 and 535 nm, respectively. The final concentrations were 100 mM phosphate buffer (pH 8.0), 3.3 mM glucose-6-phosphate, 0.3 unit/mL glucose-6-phosphate dehydrogenase, 1.0 mM NADP⁺, 2 μM DBOMF and 5 nM P450 3A4 with 0.1% DMSO. Control experiments included no DBOMF as the background and 1 μM ketoconazole as the positive control. The rates of the P450 activity were obtained from the slope of the change of the fluorescent intensity over the initial 8 min.

4b,5,6,7,8,8a-cis-Hexahydro-2-hydroxy-4b,8,8a-trimethylphenanthren-9,10-dione (OHCT). Spontaneous oxidation of HCT to OHCT was carried out in 100 mM phosphate buffer (pH 7.4) and 1.0 mM HCT in 10% acetonitrile. After incubation for 3 h at 37° C., the reaction solutions were analyzed with HPLC separation using the method described above.

Synthesis: To a solution of HCT (60 mg, 0.23 mmol) in CH₂Cl₂ (20 mL) was added triethylamine (50 mg, 0.59 mmol) and CuCl₂. 2H₂O (50 mg, 0.29 mmol) in 0.2 mL water. The reaction mixture was stirred at room temperature for 16 h and then quenched with 1 N HCI solution (10 mL). The resulting mixture was diluted with CH₂Cl₂ (100 mL) and was washed with brine (75 mL). The organic layer was collected, dried with MgSO₄ and concentrated. Flash chromatographic separation (0, 15%, 30% EtOAc in hexanes) afforded OHCT as a yellow oil (39 mg) in 62% yield. IR (film, cm⁻¹): 2936, 1717, 1674, 1609, 1497, 1449, 1310, 1225; ¹H NMR (CDCl₃, 400 MHz): δ 7.62 (d, J=2.4 Hz, 1H), 7.39 (d, J=8.4 Hz, 1H), 7.25-7.24 (m, 1H), 6.54 (s, 1H), 2.65 (s, 1H), 2.52 (br d, J=14.4 Hz, 1H), 1.58-1.54 (m, 2H), 1.39-1.30 (m, 3H), 1.17 (s, 3H), 0.94 (s, 3H), 0.39 (s, 3H); ¹³C NMR (CDCl₃, 75 MHz): δ 199.5, 181.6, 155.5, 142.8, 134.7, 126.6, 124.3, 115.7, 69.1, 42.1, 39.4, 39.3, 36.5, 35.8, 31.6, 24.5, 19.0; HRMS calcd for C₁₇H₂₀O₃Na (M+Na⁺) 295.1310, found 295.1288.

Enzyme and Cellular Studies with OHCT. The inhibitory effect of OHCT on P450 3A4 and the metabolic conversion of AFB1 with liver microsomes were studied similarly as described for HCT. The inhibitory mechanism of OHCT on P450 3A4 was revealed by a Lineweaver Burk plot of the rates obtained at various concentrations of DBOMF and OHCT with the Vivid® P450 3A4 activity assay. The P450 activity was carried out similarly as described above with concentrations of DBOMF at 0.50, 0.67, 1.0, and 2.0 μM, respectively. The rates of the P450 activity were obtained as the slope of the change of the fluorescent intensity over the initial 4 min. The inhibitory constants of OHCT were obtained from the global best fit of the mixed inhibition model by GraphPad Prism (version 4.00, GraphPad Software, San Diego, Calif.).

The cell viability MTT assay on human HepG2 cells was carried out as previously reported. Briefly, HepG2 cells were seeded at 30,000 cells/well on a 96 well plate for 4 h before the treatment with AFB1 and OHCT or ketoconazole. After incubation for 72 h, the MTT assay was performed, followed by obtaining the absorbance of each well at 570 nm with a plate reader (μQuant, BioTek Instruments, Vt.). The statistical analyses (one-way ANOVA with Dunnett's test) were performed by GraphPad Prism.

Cultures of Plasmodium falciparum. P. falciparum clone Dd2 (chloroquine IC50, 560 nM), were cultured by a method modified from that of Trager and Jensen in a 5% CO2 atmosphere at 37° C.

In vitro antiplasmodial activity: All solutions of chloroquine diphosphate, artemisinin (Sigma Aldrich), and OHCT were prepared in media RPMI 1640, ethanol and 0.1% DMSO, respectively. The OHCT solutions were checked to determine that they did not precipitate under these conditions. Parasite growth was estimated by microscopic observation of Giemsa-stained blood smears. All results presented are the means of at least three independent experiments.

OHCT action on specific parasite life cycle stages: P. falciparum cultures were synchronized with 5% sorbitol and the Percoll-Sorbitol methods. Dilutions of OHCT, chloroquine, and artemisinin were prepared. To determine the in vitro efficacies of the three antimalarial drugs in culture, serial dilutions were prepared: for artemisinin, chloroquine and OHCT at 2, 4, 8, 16, 32 and 40 μM, respectively. After synchronization, the parasites were plated at the ring stage in 96-well plates. Each plate corresponded to one of the successive 6-h periods of the P. falciparum life cycle.

### Results

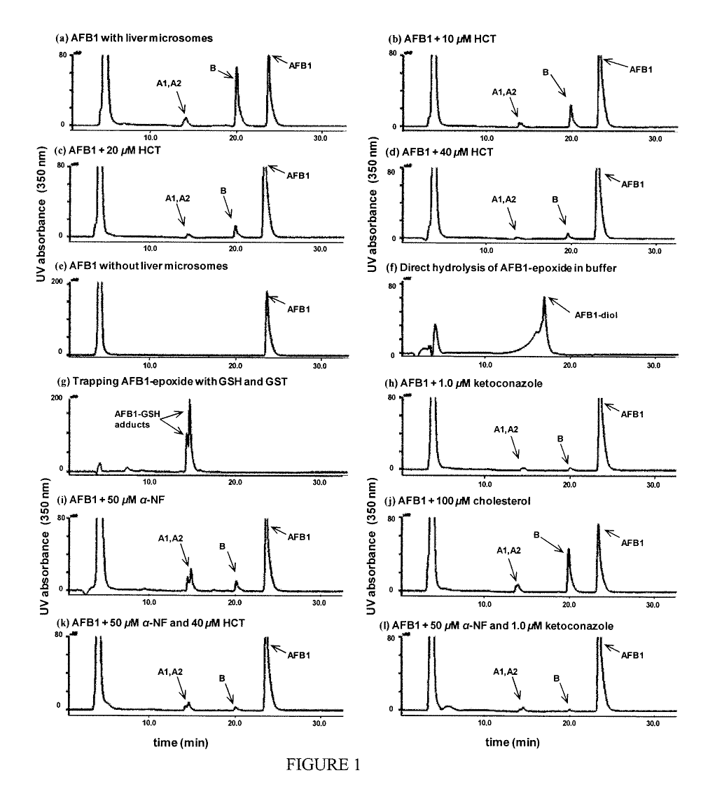

Inhibitory Effects of HCT on the Metabolic Conversion of AFB1 with Liver Microsomes and P450 3A4. The impact of HCT on the metabolic conversion of AFB1 was first investigated with pooled human liver microsomes using a NADPH regenerating system by HPLC analysis (FIG. 1). The optimal reaction condition for the AFB1 conversion and subsequent GSH trapping was to incubate the reaction mixture at 37° C. for 90 min with 50 μM AFB1 and 0.4 mg/mL liver microsomes in phosphate buffer (pH 7.4), 1.3 mM NADP⁺, 3.3 mM MgCl₂, 3.3 mM glucose-6-phosphate, 0.3 unit/mL glucose-6-phosphate dehydrogenase, 20 mM GSH, 2 mM EDTA, 0.1 mg/mL GST, and 1% DMSO. The HPLC chromatograms showed that AFB1 was converted to new signals A1, A2 and B at 13.8, 14.0, and 20.0 min, respectively, only in the presence of liver microsomes (FIG. 1a versus 1e). The appearance of these new signals concurred with the decreased intensity of the AFB1 signal at 23.0 min. The addition of HCT in the AFB1 metabolic study with liver microsomes was achieved by lyophilizing solutions of HCT in acetonitrile first and then reconstituting with the reaction solutions to a series of concentrations of HCT. As shown in FIG. 1a-d, increasing concentrations of HCT from 0, 10, 20 to 40 μM significantly decreased the formation of the signals A1, A2 and B and at the same time, increased the amount of unmetabolized AFB1 in a concentration-dependent manner.

The identity of these metabolite signals of AFB1 was next investigated with that of exo-AFB1-epoxide, the carcinogenic metabolite of AFB1. FIG. 7 illustrates that in the presence of GSH and GST, carcinogenic exo-AFB1-epoxide is converted to the GSH-AFB1 adduct while in the absence of nucleophiles, the AFB1-diol is formed from direct hydrolysis because of the high reactivity of the epoxide ring. Indeed, in our investigations we observed the hydrolysis product AFB1-diol in the KPLC analysis and confirmed with ESI-MS analysis when the exo-AFB1-epoxide was incubated directly in the phosphate buffer (pH 7.4) and 1% DMSO (FIG. 1f). However, the observed AFB1-diol had different retention time than any of the signals observed in FIG. 1a-d. On the other hand, incubation of the exo-AFB1-epoxide with 20 mM GSH and 0.1 mg/mL GST resulted in two signals at 13.8 and 14.0 min in HPLC analysis (FIG. 1g), same as A1 and A2 observed in FIG. 1a. These two signals showed an identical m/z signal at 738.16 in the MS analysis and were assigned as the stereoisomers of the GSH-AFB1 adduct plus a triethylammonium ion because triethylammonium acetate buffer was used as the aqueous eluant in the HPLC analysis. In addition, no AFB1-diol signal was observed under this condition, indicating the effective trapping of the exo-AFB1-epoxide with GSH and GST. Therefore, it was confirmed that the carcinogenic conversion of AFB1 with liver microsomes was inhibited by HCT in a concentration dependent manner.

The inhibitory effect of HCT on the metabolic conversion of AFB1 was then compared with the effect of known P450 inhibitors such as ketoconazole for P450 3A4/3A5, based on reported pathways of AFBL metabolism. We found that 1 μM ketoconazole inhibited the metabolism of AFB1 to the same extent as 40 μM HCT (FIG. 1d versus 1h). This implied that HCT had a similar inhibition target as ketoconazole on P450 3A4 primarily, due to the fact that the specific activity of P450 3A4 is much higher than that of P450 3A5 in human liver. Because only aflatoxin Q₁ and exo-AFB1-epoxide are formed from AFB1 with P450 3A4 (15) and can be specifically inhibited by ketoconazole; the HPLC signal B at 20 min, which had a higher m/z signal of 16 than that of AFB1 in ESI-MS analysis, was assigned as the aflatoxin detoxification metabolite Q₁.

Natural flavonoids, especially a-naphthoflavone (α-NF), are able to change the metabolite profile of AFB1 metabolism with P450 3A4 by enhancing the production of exo-AFB1-epoxide and reducing that of aflatoxin Q₁. Indeed, we observed that the amount of GSH-AFB1 adducts increased by 2.3 folds while aflatoxin Q₁ decreased by 6 fold in the presence of 50 μM α-NF than that with no α-NF (FIG. 1i versus 1a). On the other hand, addition of either 40 μM HCT or 1 μM ketoconazole consistently inhibited the stimulated production of exo-AFB1-epoxide and further lowed the amount of aflatoxin Q₁ (FIG. 1k-l). In addition, cholesterol was included as the structural control in the study because cholesterol has a similar carbon skeleton as that of HCT. However, no inhibitory effect was observed with cholesterol up to 100 μM (FIG. 1j), which indicated that the inhibitory effect of HCT on the metabolic conversion of AFB1 was unique to the flnctional groups and stereochemistry of HCT.

To confirm that the observed effect of HCT was not due to the inhibition of the NADPH regenerating system, we investigated the effects of HCT on the AFB1 metabolism with liver microsomes using NAPDH directly in the absence of GSH and GST. Similar inhibitory effect with 40 μM HCT was observed as with the NADPH regenerating system, which indicated that HCT does not interfere with the glucose-6-phosphate dehydrogenase or the GST activity. Therefore, it was concluded that HCT not only blocks the detoxifying conversion of AFB1 with the liver microsomes but more importantly, inhibits the formation of the key carcinogenic exo-AFB1-epoxide from AFB1.

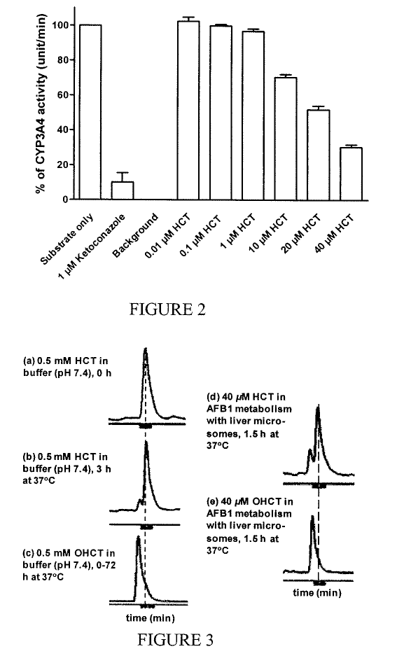

The similar inhibitory effect of 40 μM HCT and 1 μM ketoconazole on the metabolic conversion of AFB1 led us to investigate the impact of HCT on the purified P450 3A4 enzymatic activity. The enzyme assay was achieved by using a commercially available fluorescent Vivid(E activity assay with a NADPH regenerating system. The rate of P450 3A4 activity was derived from the slope of the initial change of fluorescent intensity, and the percentage activity was calculated on the basis of the rate with no inhibitors. With reference to FIG. 2, HCT decreased the P450 3A4 activity approximately by 30%, 50% and 70% with 10, 20 and 40 μM, respectively, while 1 μM ketoconazole resulted in more than 90% inhibition of the enzyme activity. Thus, these results further demonstrated that HCT acted as an inhibitor of P450 3A4 to block the metabolic conversion of AFB1 in the liver microsomes.

On the other hand, as illustrated in FIG. 3, we observed there was a slow degradation of HCT in the phosphate buffer and in the AFB1 metabolic study. Thus, the identity of the degradation product needed to be established.

OHCT as the Oxidation Product of HCT and its Inhibitory Effects on P450 3A4, the metabolism of AFB1 with Liver Microsomes, and Chemoprotection against AFB1 in Liver Cells. The degradation of HCT in the phosphate buffer was observed after incubation at 37° C. for 3 h by HPLC analysis (FIG. 3a-c). The intensity of the new signal at 29.4 min in the BIPLC chromatogram was much enhanced if 2.0 mM CuCl₂ was added in the reaction solution. Also, this degradation signal was observed in the metabolic study of AFB1 with liver microsomes at 37° C. after 1.5 h (FIG. 3d). On the other hand, no significant degradation was observed by HPLC analysis if stock solutions of HCT were stored in acetonitrile at −15° C. for several weeks. Because the degradation of HCT under aqueous conditions may complicate the inhibitory mechanism on P450 enzymes, the signal at 29 min was isolated and identified through a synthetic approach as the oxidation product OHCT.

The synthesis of the oxidized product OHCT was accomplished by treating HCT with triethylamine and CuCl₂ for 16 h, followed by a chromatographic purification as illustrated in FIG. 8 and as discussed above. A full characterization including IR, ¹H, ¹³C NMR and MS analyses revealed that OHCT was the oxidation product at the benzylic position of HCT. OHCT was further confirmed as the degradation product of HCT under aqueous conditions with the same retention time, UV spectrum and MS analysis results. In addition, the stability of OHCT under aqueous conditions was also investigated, and no degradation of OHCT was observed by HPLC analysis after incubating for 72 h at 37° C. (FIG. 3c). Not to be bound by theory, it is hypothesized that the oxidative mechanism of HCT to OHCT was possibly through the formation of an enol, followed by the nucleophilic attack of the enol to molecular oxygen, and subsequent elimination of hydroxyl ion via a second enol (FIG. 8).

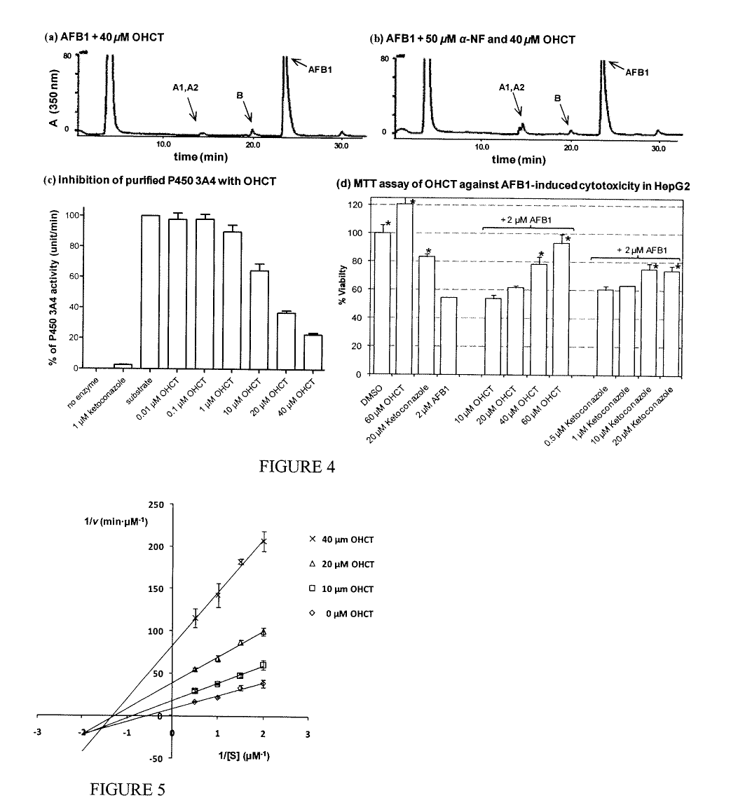

To clarify the role of OHCT in the observed inhibitory effect of HCT, the impacts of OHCT on the AFB1 conversion and the P450 3A4 were assessed similarly as with HCT. Incubation of 40 μM OHCT, 50 μM AFB1 with liver microsomes and the NADPH regenerating system in the absence or the presence of 50 μM α-NF resulted in the observation of inhibition of both GSH-AFB1 adducts and aflatoxin Q₁ in the HPLC analysis (FIG. 4a-b), similar to that of HCT. More importantly, the extent of inhibition on the GSH-AFB1 adducts from the carcinogenic activation of AFB1 with 40 μM OHCT was similar to that with HCT. For the P450 3A4 enzymatic activity, OHCT exhibited a similar 50% inhibition at 20 μM as HCT (FIG. 4c). These results implied that the observed inhibitory effects of HCT might be due to the actions of OHCT. However, HPLC analysis showed that in the metabolic of AFB1 with liver microsomes and 40 μM HCT, the area integration of HCT at 280 nm was about four times higher than that of OHCT (FIG. 3d), suggesting that HCT has a significant role in the inhibitory effects on the metabolic pathway of AFB1 and possibly, there is no difference between HCT and OHCT. On the other hand, OHCT is a better choice of inhibitors because no degradation was observed by HPLC analysis under aqueous conditions and in the metabolic study of liver microsomes (FIG. 3e).

The inhibitory mechanism of OHCT on the purified P450 3A4 was revealed with a Lineweaver-Burk plot of the enzyme rates at a combination of concentrations of the enzyme substrate DBOMF and OHCT as the inhibitor. The rates of the enzyme activity were obtained using the green Vivid(& activity assay and the data are shown in FIG. 5. The linear fit of the data points of each concentration of OHCT produced four lines converged near a single point below the X-axis, indicating a mixed inhibitory mechanism of OHCT on P450 3A4. On the basis of this result, a global best fit of these data using the mixed inhibition model (equation shown below) was performed with the GraphPad Prism to obtain Kᵢ=17.6±5.6 μM and Kᵢ′=7.6±1.5 μM, where Kᵢ is the disassociation constant of enzyme inhibitor complex and Kᵢ′ is the disassociation constant of enzyme-substrate inhibitor complex.

$$v = \frac{{V}_{max} [ S ]}{( 1 + \frac{[ I ]}{{K}_{i}} ) {K}_{m} + ( 1 + \frac{[ I ]}{{K}_{i}^{′}} ) [ S ]}$$

Finally, the chemoprotection against AFB1-induced cytotoxicity with OHCT in liver HepG2 cells was verified (FIG. 4d). Cell viability after the co-treatment of 2 μM AFB1 with 10, 20, 40 or 60 μM OHCT was assessed with MTT assay after incubation for 72 h (see supporting information). As a comparison, the chemoprotection of ketoconazole was also investigated. Consistently, OHCT showed protection against the induced cytotoxicity by AFB1 in a concentration dependent manner with more than 90% cell viability at 60 μM OHCT while OHCT alone at 60 μM exhibited no cytotoxicity. This result correlated well with the inhibitory effects of OHCT in the above liver microsomes and purified enzyme studies and implied that the mixed inhibition of the P450 3A4 activity was a chemoprotective mechanism with cis-terpenones because both MRNA and the enzyme activity of P450 3A4 are detected in HepG2 cells. In contrast, ketoconazole alone exhibited 20% cell mortality at 20 μM (FIG. 4d), which is consistent with the reported hepatotoxicity of ketoconazole. Furthermore, chemoprotection against 2 μM AFB1 in HepG2 with ketoconazole was observed at much higher concentrations (10 and 20 μM, FIG. 4d) than the 1 μM concentration used in the inhibitory assays of P450 3A4 activity and AFB1 metabolism with liver microsomes, and thus suggests that OHCT may be a preferred drug alternate for ketoconazole.

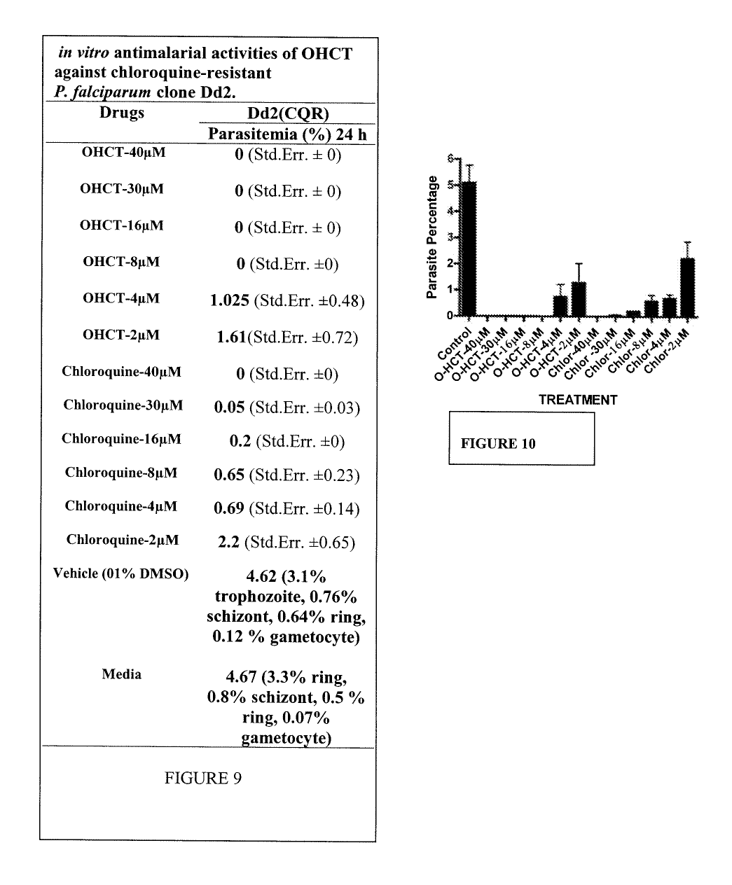

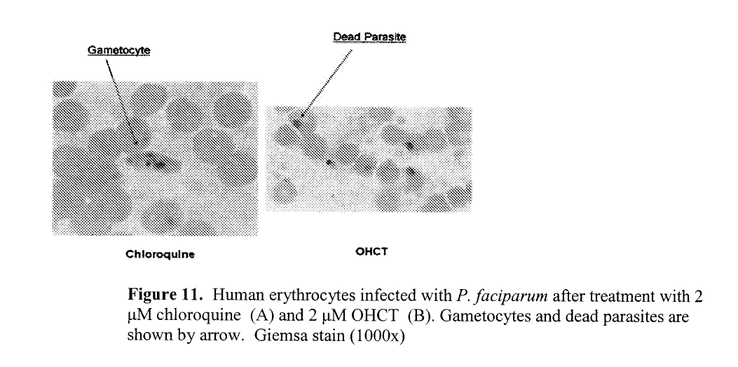

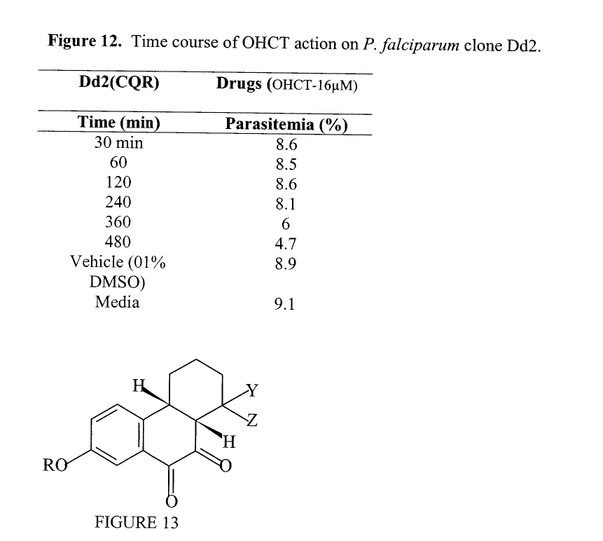

Antimalarial actions of OHCT: Experiments have been conducted which show that OHCT is effective in killing the causative agent of the most severe form of malaria, the parasite Plasmodium falciparum within 24 h (FIG. 9 and FIG. 10). OHCT has a unique structure, unlike any known antimalarial. Experiments have shown that OHCT kills all life cycle stages of Plasmodium falciparum, including the gametocyte stage that is responsible for infecting mosquitoes and transmitting disease (FIG. 11). Further, OHCT has been found to kill parasites that are resistant to the well-known antimalarial drugs, artemisinin and chloroquine (Example in FIG. 9). In a time course assay, parasitemia decreased by 50% following treatment with 16 μM OHCT for 8 h (FIG. 12). OHCT has the advantage that it exhibits no toxicity to human liver or lung cells in culture, and mhice receiving OHCT over a period of four days displayed no signs of toxicity three months after administration.

FIG. 13 illustrates OHCT as well as conjugates and derivatives thereof, where R is a hydrogen or cation such as sodium, potassium, ammonium, acetyl, etc., and where Y and Z can be the same or different and can be a substituted or unsubstituted C₁₋₆ alkyl (i.e., Y and Z are methyls in OHCT) (by “substituted” we mean the presence of moieties other than hydrogen such as halogens, ammoniums, oxygen or sulfur containing moieties, etc.). The presence of hydrogen at site R (as is the case in OHCT) makes the compound more water soluble which will be preferred in most drug applications. When used as an antimalarial, other known antimalarial compounds (e.g., chloroquine) may be conjugated to OHCT at, for example, the phenol through an ether, ester or alkyl linkage.

A subject (human or mammal) in need of antimalarial therapy or inhibition of the carcinogenic conversion of AFB1 to exo-AFB1-epoxide by noncompetitive inhibition of P450 enzymes such as 3A4 or inhibition of TCDD induced cytotoxicity will be administered a therapeutically effective amount of OHCT or conjugates or derivatives thereof. Routes of administration include, for example, intraarterial administration, epicutaneous administration, ocular administration (e.g., eye drops), intranasal administration, intragastric administration (e.g., gastric tube), intracardiac administration, subcutaneous administration, intraosseous infusion, intrathecal administration, transmucosal administration, epidural administration, insufflation, oral administration (e.g., buccal or sublingual administration), oral ingestion, anal administration, inhalation administration (e.g., via aerosol), intraperitoneal administration, intravenous administration, transdermal administration, intradermal administration, subdermal administration, intramuscular administration, intrauterine administration, vaginal administration, administration into a body cavity, surgical administration (e.g., at the location of an infection or tumor or internal injury), administration into the lumen or parenchyma of an organ, or other topical, enteral, mucosal, or parenteral administration, or other method, or any combination of the forgoing as would be known to one of ordinary skill in the art (see, for example, Remington's Pharmaceutical Sciences, 18th Ed. Mack Printing Company, 1990). The OHCT or its conjugates or derivatives will typically be formulated in a pharmaceutically acceptable composition. The compositions may be prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of active agent(s) in the formulations may vary. However, the amount of OHCT, or OHCT conjugates or derivatives, in such compositions will generally be in the range of about 1-99% by weight or by volume, with the additional constituents being in the range of 1-99% by weight or by volume. By “therapeutically effective amount” we mean an amount of an OHCT or OHCT conjugates or derivatives that kills Plasmodium falciparum and alleviates the symptoms of malaria in the case of patients suffering from malaria in the case where the patient is suffering from or suspected to be suffering from malaria. By “therapeutically effective amount” we mean an amount of an OHCT or OHCT conjugates or derivatives that inhibits the conversion of AFB1 to exo-AFB1-epoxide by p450 enzymes in the case of a patient in need of protection against AFB1 induced cytotoxicity. By “therapeutically effective amount” we mean an amount of an OHCT or OHCT conjugates or derivatives that inhibits P450 mediated carcinogenesis in the case of a patient in need of protection against potential cancer risk when treated with drugs such as estrogens, nitrocyclic hydrocarbons, aromatic amines, heterocyclic amines and polycyclic aromatic hydrocarbons or exposed to any cytotoxic substances. A therapeutically effective amount or a therapeutic effective amount can also be an amount that is given prophylactically thereby inhibiting a pathophysiological effect of AFB1 or Plasmodium falciparum infections. The amount may depend upon, for example, subject size, gender, magnitude of the associated condition or injury, age and genetic or non-genetic factors associated individual pharmacokinetic or pharmacodynamic properties of the administered molecule of the invention. For a given subject in need thereof a therapeutically effective amount or a therapeutic effective amount can be determined by those of ordinary skill in the art and by methods known to those of ordinary skill in the art.

While the invention has been described in the context of exemplary embodiments, those of skill in the art will recognize that the invention can be practiced with variation in the scope and context of the appended claims.

1. A compound having the structure: 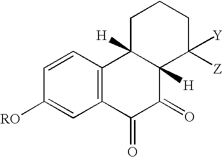
wherein R is hydrogen or a cation, and where Y and Z are the same or different and are substituted or unsubstituted C₁₋₆ alkyl.

2. The compound of claim 1 wherein R is a hydrogen and where Y and Z are both methyls.

3. The compound of claim 1 wherein R is a cation and is selected from the group consisting of sodium, potassium, substituted or unsubstituted C₁₋₆ alkyl, acetyl, and ammonium.

4. A composition, comprising
a compound having the structure 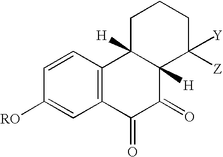
wherein R is hydrogen or a cation, and where Y and Z are the same or different and are substituted or unsubstituted C₁₋₆ alkyl; and
another constituent, wherein said compound is present at 1-99% by weight or by volume, and said another constituent is present at 1-99% by weight or by volume.

5. The composition of claim 4 wherein R in said compound is a hydrogen and wherein Y and Z in said compound are both methyls.

6. The composition of claim 4 wherein said another constituent is an aqueous fluid.

7. A method for killing Plasmodium falciparum, comprising the step of exposing Plasmodium falciparum to a sufficient quantity of a compound having the structure 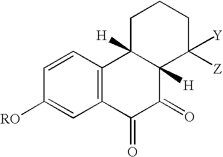
wherein R is hydrogen or a cation, and where Y and Z are the same or different and are substituted or unsubstituted C₁₋₆ alkyl.

8. The method of claim 7 wherein R is a hydrogen and where Y and Z are both methyls.

9. A method of treating or prophylactically preventing malarial infection in a subject, comprising the step of providing said subject with a therapeutically effective amount of a compound having the structure 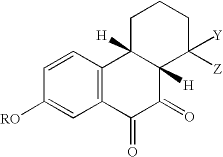
wherein R is hydrogen or a cation, and where Y and Z are the same or different and are substituted or unsubstituted C₁₋₆ alkyl.

10. The method of claim 9 wherein R is a hydrogen and where Y and Z are both methyls.

11. A method of inhibiting aflotoxin B1 induced cytotoxicity in a subject, comprising the step of providing said subject with a therapeutically effective amount of a compound having the structure 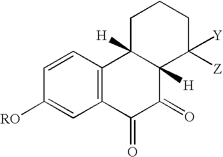
wherein R is hydrogen or a cation, and where Y and Z are the same or different and are substituted or unsubstituted C₁₋₆ alkyl.

12. The method of claim 11 wherein R is a hydrogen and where Y and Z are both methyls.

13. A method of inhibiting 2,3,7,8 tetrachlorodibenzo-p-dioxin (TCDD) induced cytotoxicity in a subject, comprising the step of providing said subject with a therapeutically effective amount of a compound having the structure 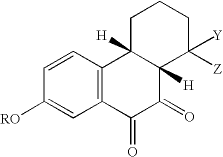
wherein R is hydrogen or a cation, and where Y and Z are the same or different and are substituted or unsubstituted C₁₋₆ alkyl.

14. The method of claim 13 wherein R is a hydrogen and where Y and Z are both methyls.
